(19) United States
(12) Patent Application Publication
(10) Pub. No.: US 2008/0260661 A1
(43) Pub. Date: Oct. 23, 2008

(54) Aminobutadiene-Based Uv Screens

(75) Inventors: Sebastianus Gerardus Johannes Maria Kluijtmans, Zeist (NL); Akihiko Ikegawa, Tilburg (NL); Jan Bastiaan Bouwstra, Bilthoven (NL)

Correspondence Address: FOLEY AND LARDNER LLP;SUITE 500, 3000 K STREET NW, WASHINGTON, DC, 20007, US

(73) Assignee: Fuji Photo Film B.V.

(21) Appl. No.: 11/573,120

(22) PCT Filed: Aug. 10, 2005
(86) PCT No.: PCT/NL05/00579

§ 371 Date: Aug. 29, 2007

(30) Foreign Application Priority Data
Aug. 11, 2004 (EP) 04077274.1

(51) Int. Cl. A61K 8/40; A61Q 17/04

(52) U.S. Cl. 424/59

(57) ABSTRACT

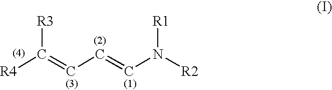

The invention relates to the use of an UV-absorbing compound for the preparation of a cosmetic composition for protection against UV radiation said compound being represented by the general formula (I) wherein less than 10% of the total absorption between 250 and 600 nm of said UV-absorbing compound according to formula (I) is above 400 nm. The invention also relates to sunscreen compositions comprising the UV-absorbing compound according to formula (I).

### FIELD OF THE INVENTION

The present invention is in the field UV-absorbing compounds, in particular useful in sunscreen compositions.

### BACKGROUND OF THE INVENTION

The detrimental effects of exposing the skin to UV light are manifold and are well documented in the prior art. It has been long recognized that UV-B radiation, with a wavelength of 290 to 315 nm, causes erythema or sunburn. It was not until around 1980 that it was discovered that UV-A radiation, with a wavelength from 315 to 400 nm, causes phototoxic and photochemical reactions.

While about 70% UV-B radiation is blocked by the outer skin or stratum corneum, this is not the case for UV-A radiation, which can subsequently penetrate deep into the living dermis. A well known destructive effect of UV-A is oxidative stress. Superoxide, which is formed by UV-A radiation, can release iron from ferritin, an iron-storage protein located in fibroblasts in the skin (Pourzand et al, Proc. Natl. Acad. Sci. USA, June 1999, Vol 96, p. 6751-56). The role of iron in the Fenton or Haber Weis reaction resulting in the production of highly destructive hydroxyl radicals and hydrogen peroxide is well known. Also other metal ions like copper-ions have been reported to catalyze the formation of oxygen radicals. The role of these destructive products in damaging DNA is well known as for example described by Sestili et al (Free Radical Biology & Medicine [US], Jul. 15, 1998, 25, [2] p. 196-200).

The normal biochemical protection by enzymes like superoxide dismutase is not sufficient to effectively stop the reaction induced by UV-A radiation. Hence the necessity to protect the skin from these harmful effects is still mandatory. Other oxidative stress phenomena are damaging of collagen resulting for example in accelerated skin aging and white spots, or damaging of cell walls by lipid peroxidation.

The use of organic UV absorbers for sunscreen applications is widely known.

For example, U.S. Pat. No. 5,945,091 discloses particular enamine compounds that absorb in the range of approximately 320-380 nm and that are primarily used as UV-A filters in cosmetic and pharmaceutical compositions to protect the human skin for UV radiation.

U.S. Pat. No. 6,238,648 discloses 4,4-diaryl-butadienes that absorb in the UV-A region with high extinction. Table II of U.S. Pat. No. 6,238,648 discloses that preferred compounds have an absorption in the range of about 330 nm to about 350 nm.

EP A 895.776 discloses particular N-fenoxy enamine compounds that can be used in compositions to protect human hair or skin against UV radiation. The compounds absorb around 320 to 340 nm.

U.S. Pat. No. 2,617,827 discloses the synthesis of bis-1,4-dialkylamino-1,3-butadienes but mentions no applications for the compound.

U.S. Pat. No. 4,045,229 discloses photographic application of 1-amino-4-cyano-1,3-butadiene as a UV-absorbing compound but is silent with respect to other applications. Further, such compounds are less preferred for cosmetic applications due to the presence of harmful groups such as cyanide.

U.S. Pat. No. 4,195,999 and U.S. Pat. No. 4,309,500 also discloses photographic applications of 1-amino-1,3-butadiene derivatives.

U.S. Pat. No. 4,950,467 describes sunscreen compositions which contain certain 5-phenyl pentadienoate esters which act as broadband UV filters. These esters have too short wavelength too act as an adequate UV-A absorber.

WO 2004/006878 discloses merocyanine derivatives for use in cosmetics for protection against UV radiation, but provides no teachings about the benefits or drawbacks of the numerous listed structures.

A disadvantage of UV absorbing compounds is that they are unstable under UV light. UV exposure can cause photochemical reactions that destroy the UV absorbing compound thereby reducing the protection against UV radiation. This is especially a disadvantage of UV absorbers of the butadiene-type, which have a low stability which makes them less attractive for cosmetic applications. Another drawback of the compounds from the prior art is that they are often highly coloured and therefore cosmetically unacceptable.

There remains therefore still a need for UV filters which provide sufficient direct protection from sunlight by blocking the radiation while being stable under UV light radiation and being cosmetically or aesthetically acceptable and being fully transparent for the visible light radiation.

### SUMMARY OF THE INVENTION

It is an object of this invention to provide stable, highly efficient, UV-absorbing compounds.

It is a further object of the invention to provide a sunscreen composition comprising a UV-absorbing compound, which is substantially transparent in the visible light region.

It is also an object of the invention to provide such UV-absorbing compounds with a minimum risk of immunological or allergic side reactions.

Surprisingly it was found that certain aminobutadienes having carefully selected structures provide highly efficient colorless UV absorbing compounds that are stable under UV irradiation conditions and do not release harmful substances under UV-irradiation.

Thus the invention relates to a UV-absorbing compound for the preparation of a cosmetic composition for protection against UV radiation said compound being represented by the general formula (I):

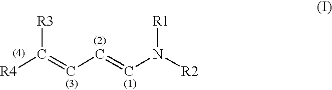

wherein R₁ and R₂, which may be the same or different, each represents a hydrogen atom, an alkyl group having 1-20 carbon atoms, or an aryl group having 6-20 carbon atoms, provided that R₁ and R₂ do not simultaneously represent hydrogen atoms, optionally R₁ and
R₂ can combine with each other, or one of R₁ and R₂ can combine with C(1) and form a cyclic amino group which is optionally interrupted by an —O— or by an —NH—; each of R₁ and
R₂ may be substituted by one or more carboxylic acid moieties;
R₃ represents a carboxyl group, —COOR₅, —CONHR₅, —CN or —SO₂R₆, and if R₁ or R₂ form a cyclic aminogroup with C(1), R₃ may also be a —COR₅ group;
R₄ represents a carboxylic group, —COOR₅, —CONHR₅, —CN or —SO₂R₆, and if R₁ or R₂ form a cyclic aminogroup with C(1), R₄ may also be a —COR₅ group;
R₅ and R₆, which may be the same or different, each represents an alkyl group having 1-20 carbon atoms or an aryl group having 6 to 20 atoms; optionally R₅ and R₆ can combine and form a ring structure which is optionally substituted and which optionally comprises N, O and/or carbonyl groups; and
wherein less than 10% of the total absorption between 250 and 600 nm of said UV-absorbing compound according to formula (I) is above 400 nm.

Further, the invention also relates to the use of a UV absorbing compound of the invention for the preparation of a cosmetic composition for protection against UV radiation, in particular UV-A radiation.

### DETAILED DESCRIPTION OF THE INVENTION

According to the present invention, the alkyl groups of general formula I may be branched, linear or cyclic. Suitable examples are methyl, ethyl, n- and i-propyl, n-, s- and t-butyl, n-pentyl, n-hexyl, cyclohexyl, dodecyl and the like.

According to the present invention, aryl groups may be phenyl, alkaryl or arylalkyl groups. Obviously, alkaryl groups and arylalkyl groups have C₇-C₂₀ carbon atoms. Suitable examples of alkaryl groups are 4-methylphenyl, 2,4-dimethylphenyl and the like. Suitable examples of arylalkyl groups are phenylmethyl, 4-methylphenylmethyl, 3-phenyl-2-propyl ant the like. The aryl groups also encompass heterocyclic aryl groups, e.g. pyridinyl, 4-methyl-pyridinyl and the like.

According to the invention, the cyclic amino group contains 3 to 8 carbon atoms and 1 to 3 nitrogen atoms, preferably 3 to 6 carbon atoms and 1 nitrogen atom, wherein a carbon atom of the cyclic amino group may be replaced by an oxygen atom. The cyclic amino group may be saturated or unsaturated, although it is preferred that the cyclic amino group is saturated. Suitable examples of the cyclic amino group are piperidinyl, 2-piperidyl, morfolinyl, imidazolidinyl and the like.

The UV-absorbing compound according to formula (I) is for brevity depicted in such a way that the R₁R₂N group and the R₄ group are in the Z,Z-configuration. However, the present invention also encompasses the other possible isomers, i.e. Z,E and E,Z.

The present invention is related to strong UV radiation absorbing compounds that can be used in compositions to protect the human skin or hair from the detrimental effects of exposure to sunlight.

Although some attempts were made to popularise the use of coloured UV protecting agents, for cosmetic reasons there is a preference for colourless UV-protecting agents. Preferably, the UV-absorbing compound according to the present invention has an absorption maximum between 340 and 380 nm, more preferably between 370 and 375 nm, to provide optimal protection from UV-A radiation. Absorption maxima at lower wavelengths give too low absorption in the UV-A range (about 320 to about 400 nm) while absorption maxima at higher wave lengths result in an undesired yellow to orange colouring of the UV absorbing compound. Thus the UV absorbing compounds of this invention have less than 10% of their total absorption between 250 and 600 nm above 400 nm.

The UV-absorbing compound according to formula (I) is considered to encompass any equivalent of 4-amino-1,3-butadiene derivatives that may be modified or substituted but has substantially the same basic structure and still has the property of retaining the high absorption. Such equivalents can be easily imagined by the skilled person. Examples of equivalents of 4-amino-1,3-aminobutadiene (I) are found for example in U.S. Pat. No. 4,045,229, U.S. Pat. No. 4,195,999 and U.S. Pat. No. 4,309,500, all incorporated by reference herein.

Groups R₃ and R₄ of the general formula (I) selected in such a way that the absorption maximum of the total structure is within the preferred range of 340 to 380 nm. The choice of R₃ and R₄ is dependant of the selection for R₁ and R₂.

The selection criteria can be defined for three cases, in which R₁ to R₆ are as defined for general structure (I)

Case 1: If R₁ and R₂ do not form a ring structure with each other or do not form a cyclic amino group together with C(1), then R₃ is a carboxyl group, —COOR₅, —CONHR₅, —CN or —SO₂R₆; and R₄ is a carboxyl group, —COOR₅, —CONHR₅, —CN or —SO₂R₆. Preferably, R₃ is then a carboxyl group, —COOR₅, —CONHR₅ or —SO₂R₆; and R₄ is then a carboxyl group, —COOR₅, —CONHR₅ or —SO₂R₆. Obviously, a carboxyl group is understood as being a —COOH group. More preferably, at least one of R₃ and R₄ is an —SO₂R₆ group, wherein it is preferred that R₅ is an alkyl group having 1-20 carbon atoms, preferably 1 to 8 carbon atoms, and R₆ is an aryl group having 6 to 20 carbon atoms.

Preferably, R₅ and R₆ do not form a ring structure since this can result in a bathochromic shift of the absorption maximum. R₃ or R₄ are not a —COR group, since these also induce an undesired bathochromic shift. The use of cyanide groups can result in a desired absorption maximum, but these are less preferred due to the release of harmful cyanide under heat and/or UV-exposure.

Table 1 (1.1i-1.5i) shows, without being limiting, examples of structures according to this preferred embodiment of the invention while structures 1.6r-1.13r are examples of undesired high absorption maxima.

TABLE 1

| | General structure | |
|---|---|---|
| | 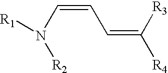 | Absorptionmaximum(nm) |
| 1.1i | 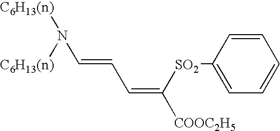 | 373 |
| 1.2i | 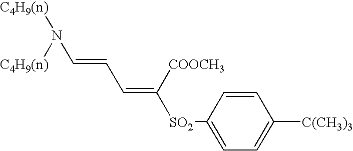 | 373 |
| 1.3i | 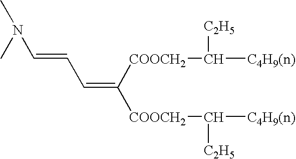 | 374 |
| 1.4i | 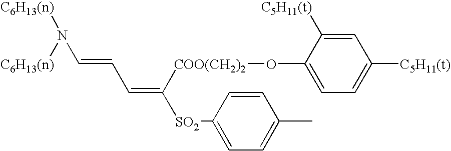 | 374 |
| 1.5i | 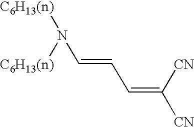 | 378 |
| 1.6r | 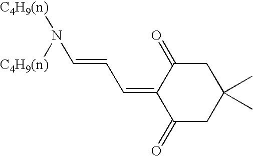 | 389 |
| 1.7r | 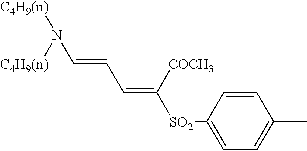 | 385 |
| 1.8r | 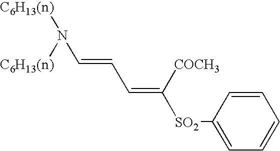 | 385 |
| 1.9r | 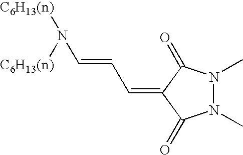 | 387 |
| 1.10r | 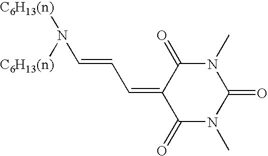 | 388 |
| 1.11r | 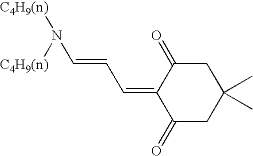 | 389 |
| 1.12r | 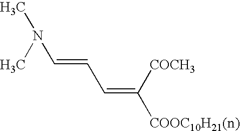 | 394 |
| 1.13r | 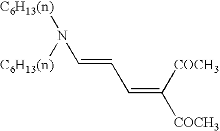 | 395 |

Case 2: R₁ and R₂ together form a saturated heterocyclic group together with the nitrogen to which R₁ and R₂ are attached, said heterocyclic group having 3 to 8 carbon atoms, preferably 3 to 6 carbon atoms, wherein one or two carbon atoms may be replaced by an oxygen atom, an NH group or an NR₇ group, wherein R₇ represents a C₁-C₁₀ alkyl group;

R₃ is a carboxyl group, —COOR₅ or —CONHR₅, —CN or —SO₂R₆; and
R₄ is a carboxyl group, —COOR₅ or —CONHR₅, —CN or —SO₂R₆.

Preferably, R₃ is a carboxyl group, —COOR₅ or —CONHR₅, or —SO₂R₆ and R₄ is a carboxyl group, —COOR₅ or —CONHR₅, or —SO₂R₆.

More preferably, at least one of R₃ and R₄ is a —SO₂R₆ group, wherein it is preferred that R₅ is an alkyl group having 1-20 carbon atoms, preferably 1 to 8 carbon atoms, and R₆ is an aryl group having 6 to 20 carbon atoms.

More preferably, the saturated heterocyclic group is a six or five membered ring. Optionally, in the ring one carbon atom may be replaced by an oxygen atom or (substituted) nitrogen atom as disclosed above. A sulphur atom as additional hetero atom is not preferred due to the bathochromic shift of the absorption maximum.

Cyanide groups can result in a desired absorption maximum, but these are less preferred due to the release of harmful cyanide under heat and/or UV-exposure.

Structure 2.1i shown in Table 2 is an example of a structure according to this preferred embodiment of the invention while structures 2.2r-2.3r are examples of undesired high absorption maxima.

Case 3: One of R₁ and R₂ forms a saturated heterocyclic ring structure together with carbon atom C(1) and the nitrogen atom to which R₁ and R₂ are attached, said heterocyclic group having 3 to 8 carbon atoms, preferably 3 to 6 carbon atoms, wherein one or two carbon atoms may be replaced by an oxygen atom, an NH group or an NR₇ group, wherein

R₇ represents a C₁-C₁₀ alkyl group;
R₃ is a carboxyl group, —COOR₅ or —CONHR₅, —CN or —SO₂R₆; and
R₄ is a carboxyl group, —COOR₅ or —CONHR₅, —CN or —SO₂R₆.

Preferably, R₃ is a carboxyl group, —COORS, —CONHR₅ or SO₂R₆ and R₄ is a carboxyl group, —COORs, —CONHR₅, or —SO₂R₆.

More preferably, at least one of R₃ and R₄ is an —SO₂R₆ group, wherein it is preferred that R₅ is an alkyl group having 1-20 carbon atoms, preferably 1 to 8 carbon atoms, and R₆ is an aryl group having 6 to 20 carbon atoms.

Preferably the saturated heterocyclic ring is a six- or five membered ring. The ring may contain an additional oxygen or (substituted) nitrogen atom as disclosed above. A most preferred heterocyclic ring is tetrahydro-3,4,4-trimethyl oxazole-2-yl (cf. for example compound 3. In in Table 3). A sulphur atom as additional hetero atom is not preferred due to the bathochromic shift of the absorption maximum. If the ring structure does not contain a second hetero atom, the selection criteria of case 1 apply. If, for example, an oxygen is present adjacent to carbon C(1), more preferably if the heterocyclic ring is tetrahydro-3,4,4-trimethyl oxazole-2-yl, at least one of R₃ and R₄ is a —COR₅ or —COR₆. Preferably the remaining R₃ or R₄ is then an —SO₂R₅ group in which this R₆ is an aryl group having 6 to 20 carbon atoms.

Structures shown in Table 3 (3.1i-3.6i) are examples of structures according to the invention while structures 3.7r-3.8r are examples of undesired high absorption maxima.

TABLE 2

| | General structure | |
|---|---|---|
| Table 2 | 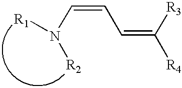 | Absorptionmaximum(nm) |
| 2.1i | 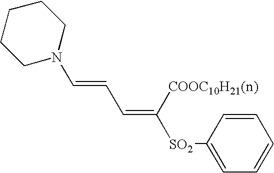 | 375 |
| 2.2r | 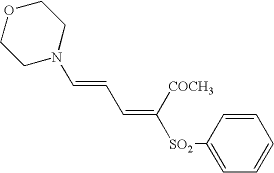 | 383 |
| 2.3r | 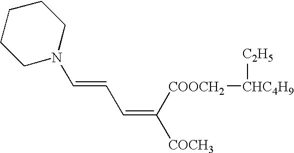 | 395 |

TABLE 3

| | General structure | |
|---|---|---|
| Table 3 | 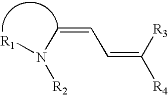 | absorptionmaximum(nm) |
| 3.1i | 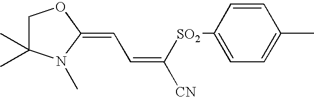 | 361 |
| 3.2i | 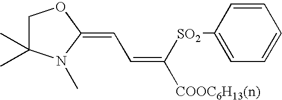 | 362 |
| 3.3i | 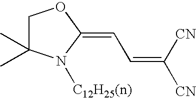 | 372 |
| 3.4i | 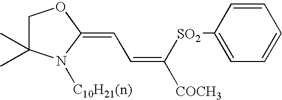 | 373 |
| 3.5i | 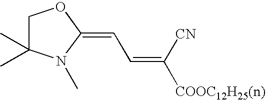 | 374 |
| 3.6i | 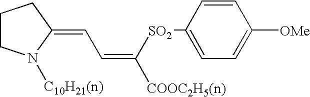 | 375 |
| 3.7r | 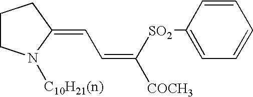 | 392 |
| 3.8r | 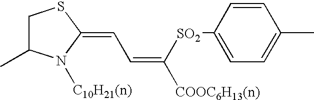 | 392 |

Absorption maxima of the structures in Tables 1 to 3 are measured in methanol, or if not soluble in methanol, in ethanol.

Of the compounds of the formula (I), those represented by the following general formula (II) are more preferred

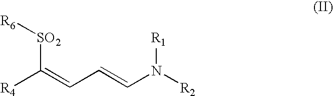

wherein R₁, R₂, R₄ and R₆ have the same meaning as in general formula (I)

Even more preferred compounds are those according to formula (I) wherein:

R₁ and R₂ are independently selected from the group consisting of hydrogen atoms, alkyl groups having 1 to 20 carbon atoms and alkylaryl groups having 6 to 20 carbon atoms, provided that R₁ and R₂ do not simultaneously represent hydrogen atoms;
R₃ and R₄ are independently selected from the group consisting of carboxylic groups, —COOR₅, —CONHRS, and —SO₂R₆, provided that R₃ or R₄ is —SO₂R₆;
R₅ and R₆ are independently selected from the group consisting of alkyl groups having 1 to 20 carbon atoms and aryl groups having 6 to 20 atoms; and wherein the total number of carbon atoms of R₁, R₂ and R₅ is not more than 8.

For the even more preferred compounds, it is preferred that R₅ is an alkyl group having 1-20 carbon atoms and R₆ is an aryl group having 6 to 20 carbon atoms.

In one embodiment the UV-absorbing compound according to the invention has the following structure formula (UV0)

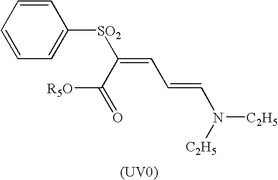

wherein R₅ has the same meaning as in general formula I. More preferably, R₅ is an alkyl group, even more preferably a linear alkyl group. In a particular embodiment R₅ represents ethyl.

Hereinafter the compound UV0 wherein R₅ is ethyl is referred to as UV1.

It was found that the —SO₂R₆, group has a stabilising effect on the UV-absorbing compounds.

In a preferred embodiment the UV-absorbing compound absorbs less than 10% of its total absorption above 400 nm. This means that the UV-absorbing compound is substantially transparent for visible light, i.e. it is essentially colourless. In a preferred embodiment the UV-absorbing compound has 75% or more of its total absorption in the UV-A region between 315 and 400 nm. Thus the invention relates to a UV-absorbing compound as defined above wherein less than 10% of the total absorption between 250 and 600 nm of said UV-absorbing compound is above 400 nm. Also the invention relates to a UV-absorbing compound as defined above wherein at least 75% of the total absorption between 250 and 600 nm of said UV-absorbing compound is between 315 and 400 nm.

The UV-absorbing compound can be advantageously used for the preparation of cosmetic or sunscreen compositions to protect the skin or hair from UV radiation. Such sunscreen compositions comprise 0.1 to 15.0 wt. %, preferably 0.1 to 10.0 wt. % and more preferably 0.1 to 5.0 wt. % of the UV-absorbing compound according to formula (I), based on the total weight of the sunscreen composition.

Various forms of cosmetic compositions for skin protection comprising UV-absorbing compounds are available in the market as lotions, emulsions, creams, milks, gels and the like. These may contain oil and/or alcohol. Also aerosols or sticks are known to be used. All such forms of cosmetic compositions may function as a medium to apply the UV absorbing compound.

One skilled in the art will be able to select suitable cosmetically and dermatologically acceptable carriers that can be used in the sunscreen composition of the invention, in particular in combination with the UV-absorbing compound according to formula (I).

The sunscreen comprising the UV-absorbing compound according to formula (I) of the invention may contain one or more further, conventional, UV-absorbing compounds. This can be a UV-A, UV-B or broadband UV absorbing compound as described in for example EP A 1.055.412, incorporated by reference herein. Other additives as applied in the art may also be used.

The cosmetic compositions of the present invention may contain in addition to the UV-absorbing compound various adjuvants conventionally present in cosmetic compositions of this type for example hydrating agents, emollients or thickening agents, surfactants, preservatives, perfumes, dyes, and the like.

In one aspect the invention relates to a sunscreen composition comprising less than 15 wt %, preferably less than 10 wt %, more preferably less than 5 wt % UV-absorbing compound according to formula (I) of the invention.

### EXAMPLE 1

### Stability of Aminobutadienes of General Formula (II)

The aminobutadiene compounds were applied in an acceptable sunscreen formulation and coated on a transparent support with a wet thickness of 24 μm. The concentrations of aminobutadiene compounds were chosen to reach an absorption at their absorption maximum (λmax) of 1.0 absorption units (A.U.) after drying (2 hrs at 40° C.). The samples were exposed to Xe-light (0.25 W/m2, Atlas) for 6 hrs after which the absorption was measured again, using a Hewlett Packard diode array spectrophotometer. Of each sample amount of absorption left after 6 hrs was compared to a control sunscreen formulation on a transparent support, that was not exposed to Xe-light. The results shown in table 4, below, clearly indicate the beneficial effect of the presence of the SO₂ group on the light stability of the aminobutadiene UV-absorbers. Of the tested structures, all formulations had a colourless appearance after application to the transparent substrate, except for structure 1.12r.

TABLE 4

| | | Sta- |
|---|---|---|
| | Sunscreen formulation | bility |
| UV-1 | 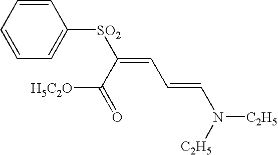 | + |
| 1.5i | 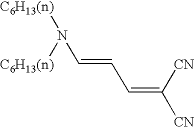 | − |
| 1.12r | 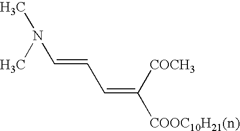 | − |
| 1.2i | 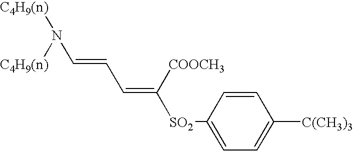 | + |
| 2.1i | 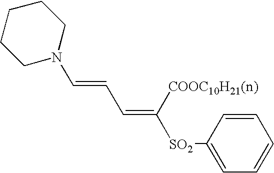 | + |

1-18. (canceled)

19. A cosmetic composition for protection against UV radiation comprising a UV-absorbing compound represented by formula (I): 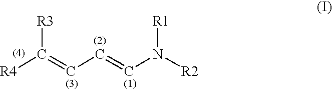
wherein R₁ and R₂ are independently selected from the group consisting of hydrogen atoms, alkyl groups having 1 to 20 carbon atoms and alkylaryl groups having 6 to 20 carbon atoms, provided that R₁ and R₂ do not simultaneously represent hydrogen atoms; R₃ and R₄ are independently selected from the group consisting of carboxylic groups, —COOR₅, —CONHR₅ and —SO₂R₆, provided that at least one of R₃ or R₄ is —SO₂R₆; R₅ and R₆ are independently selected from the group consisting of alkyl groups having 1 to 20 carbon atoms and aryl groups having 6 to 20 carbon atoms; and wherein the total number of carbon atoms of R₁, R₂ and R₅ is not more than 8.

20. The cosmetic composition according to claim 19, wherein less than 10% of the total UV radiation absorbed between 250 nm and 600 nm is above 400 nm.

21. The cosmetic composition according to claim 19, wherein R₆ is an aryl group having 6 to 20 carbon atoms.

22. The cosmetic composition according to claim 20, wherein R₆ is an aryl group having 6 to 20 carbon atoms.

23. The cosmetic composition according to claim 19, wherein R₅ is an alkyl group having 1 to 20 carbon atoms.

24. The cosmetic composition according to claim 20, wherein R₅ is an alkyl group having 1 to 20 carbon atoms.

25. The cosmetic composition to claim 23, wherein R₅ is an alkyl group having 1 to 8 carbon atoms.

26. The cosmetic composition to claim 24, wherein R₅ is an alkyl group having 1 to 8 carbon atoms.

27. The cosmetic composition according to claim 23, wherein the alkyl group is a linear alkyl group.

28. The cosmetic composition according to claim 25, wherein the alkyl group is a linear alkyl group.

29. The cosmetic composition according to claim 19, wherein R₁ and R₂ together form a saturated heterocyclic group together with the nitrogen to which R₁ and R₂ are attached, said heterocyclic group having 3 to 8 carbon atoms, wherein one or two carbon atoms may be replaced by an oxygen atom, an NH group or an NR₇ group, wherein R₇ represents a C₁-C₁₀ alkyl group.

30. The cosmetic composition according to claim 19, wherein one of R₁ and R₂ forms a saturated heterocyclic ring structure together with carbon atom C(1) and the nitrogen atom to which R₁ and R₂ are attached, said heterocyclic group having 3 to 8 carbon atoms, wherein one or two carbon atoms may be replaced by an oxygen atom, an NH group or an NR₇ group, wherein R₇ represents a C₁-C₁₀ alkyl group.

31. The cosmetic composition according to claim 30, wherein the heterocyclic group has 3 to 6 carbon atoms.

32. The cosmetic composition according to claim 31, wherein the heterocyclic group has 3 to 6 carbon atoms.

33. The cosmetic composition according to claim 19, wherein at least 75% of the total UV radiation absorbed between 250 nm and 600 nm is between 315 nm and 400 nm.

34. The cosmetic composition according to claim 21, wherein at least 75% of the total UV radiation absorbed between 250 nm and 600 nm is between 315 nm and 400 nm.

35. The cosmetic composition according to claim 23, wherein at least 75% of the total UV radiation absorbed between 250 nm and 600 nm is between 315 nm and 400 nm.

36. The cosmetic composition according to claim 19 comprising 0.1 to 15 wt % of the UV-absorbing compound.

37. The cosmetic composition according to claim 19 comprising 0.1 to 15 wt % of the UV-absorbing compound.

38. The cosmetic composition according to claim 21 comprising 0.1 to 15 wt % of the UV-absorbing compound.

39. The cosmetic composition according to claim 23 comprising 0.1 to 15 wt % of the UV-absorbing compound.

40. The cosmetic composition according to claim 30 comprising 0.1 to 15 wt % of the UV-absorbing compound.

41. The cosmetic composition according to claim 31 comprising 0.1 to 15 wt % of the UV-absorbing compound.
